(19) United States
(12) Patent Application Publication
(10) Pub. No.: US 2023/0248883 A1
(43) Pub. Date: Aug. 10, 2023

(54) MEDICAL DEVICE AND METHOD OF FABRICATION THEREOF

(71) Applicants: CSEM CENTRE SUISSE D'ELECTRONIQUE ET DE MICROTECHNIQUE SA - RECHERCHE ET DÉVELOPPEMENT, Neuchâtel (CH); UNIVERSITÄT BERN, Bern (CH)

(72) Inventors: Angélique LUU-DINH, Mulhouse (FR); Irena ZUBAK, Bern (CH)

(21) Appl. No.: 18/014,312

(22) PCT Filed: Jul. 9, 2021
(86) PCT No.: PCT/EP2021/069219

§ 371 Date: Jan. 3, 2023

(30) Foreign Application Priority Data
Jul. 17, 2020 (EP) 20186436.0

(51) Int. Cl. A61L 27/50; A61B 17/11; A61L 27/26; A61L 27/58; A61L 27/48

(52) U.S. Cl. CPC A61L 27/50; A61B 17/11; A61L 27/26; A61L 27/58; A61L 27/48; A61B 2017/00884

(57) ABSTRACT

Disclosed is a medical device for use in surgery including at least one structured substrate including a plurality of structures, at least a portion of the plurality of structures being a plurality of ridges having each a length that is at least 5 times greater than their width. The ridges have at least two different cross-sections and have swellable properties, for example in the presence of water. The medical device includes at least a second substrate, including a second plurality of structures, the first and second plurality of structures being arranged to interlock when the first substrate is pressed against the second substrate in presence of water.

### TECHNICAL FIELD

The invention relates to the field of surgical practices and in particular complex dural reconstruction and to the texturization of coatings capable to ensure a tight and hermetic cohesion of the dural substitutes and improve watertight dural closure. More precisely the invention relates to a medical device having two interlockable elements comprising textured coatings.

### BACKGROUND OF THE ART

In brain or spinal cord surgery the dura mater has to be opened. At the end of the procedure the dura is closed using different techniques depending on the anatomical localization. But when suture of the edges of the remaining dura is not possible, the defect is usually closed using the inlay-overlay technique where one layer of dural substitute is placed beneath and one above the dural defect.

For cerebral and spinal cord surgery, repair of the dura mater can present technical challenges. In particular, insufficient closure of complex dural defects commonly results in cerebrospinal fluid (CSF) leakage. This can lead to complications such as meningitis and may require revision surgeries. Multiple options exist for the repair of a dural defect such as surgical sutures with or without application of sealants/adhesives, duroplasty with autologous pericranium or non-autologous dural substitutes, which are usually made of highly processed equine or bovine collagen. More recently, entirely synthetic and absorbable dural substitutes have received Food and Drug Administration (FDA) approval. This new class of products has the advantage of reduced cortical adhesion and decreased inflammatory reactions compared to biologic materials. However, for complex dural reconstructions where suturing or application of sealants is impossible the available dural substitutes often dislocate. To overcome the dislocation of the patches there are inventions that engineered mechanical or chemical adhesive solutions to provide more adherence.

For example, U.S. Ser. No. 14/092,015 adhesive articles containing a combination of surface micropatterning such as micro-protrusions and a coating of adhesive glue and methods of making and using thereof. The articles proposed herein exhibit a 90° pull off adhesion of about 1.5 N/cm². The microtopography described doesn't improve substantially the adhesion strength compared to article without microtopography but intend to reduce the quantity of adhesive glue coated and decrease the toxicity of the article.

In KR101867058B, a device is described using hydrogel and manufacturing method of the same. The device describes two surfaces made of hydrogel and containing complementary protrusions arranged in regular arrays. The two surfaces are pressed together such as the protrusions interlock. The interconnected protrusions exhibit a strong and reversible interlocking adhesion in wet or underwater conditions based on the hydration-triggered swelling behavior of the hydrogel polymer. Adhesion for of 80N/cm² in shear force and 14N/cm² in pull force are reported after prolonged immersion for 20 h in water. This concept may be efficient, however the very tiny dimensions of the protrusions array and the prolonged time to achieve a good adhesion make the method practicably impossible to implement in surgical practices.

### SUMMARY OF THE INVENTION

It is an objective of the invention to provide a medical device to be used in surgical practices, more precisely in dural reconstruction. The device proposes solution to limitations of solutions of prior art.

The invention proposes a method providing a solution that can be implemented on existing dural substitute consisting in a simple texturization of a swellable material already Food and Drug Administration (FDA) approved which is improving shelf bonding strength and avoid dislocation of the dural substitute that may also serve as a platform technology in other surgical disciplines.

More precisely the invention relates to a medical device for use in surgery comprising at least a first substrate and at least a second substrate, said at least first substrate comprising a first surface, comprising a first plurality of structures, said at least second substrate comprising a second surface, comprising a second plurality of structures, said first and second plurality of structures being arranged to interlock when said first surface is pressed against said second surface.

At least a portion of said first plurality of structures and/or at least a portion of said second plurality of structures is a plurality of ridges having each a length that is at least 5 times greater than their width, said width and length being defined in a plane parallel to said first and/or second surface. The ridges have at least two different cross-sections defined in planes parallel to said first or respectively said second surface. Said first and second surface may be curved surfaces. The ridges have swellable properties in presence of a wet environment. The swellable properties ensures a better adhesion and/or hermeticity and avoid fluid leakage.

Advantageously a wet environment may be a natural wet environment such as the presence of cerebrospinal fluid (CSF), blood, water or humidity at the place of the closure to be realized. The wet environment may also be enhanced locally by applying a flow of a liquid, such as water, physiological saline solutions, or other solutions used as irrigation fluids during neurosurgical procedure. Such solutions can contain pharmaceutical agents as well as other additives such as tissue healing promoting agents, cell adhesion promoter and the likes.

In order to obtain a strong adhesion faster, and to obtain a better initial hermiticity, other liquids, ions and/or vapors may be used to increase the swelling kinetics of the swellable material of the ridges. For example, physiological saline and other solutions used as irrigation fluids during neurosurgical procedure may be used.

The proposed device of the invention is more efficient, practical and reliable than prior art solutions. Furthermore, the proposed device may be realized by a high productivity process as the swellable structured layer may be tailored during the device manufacturing. Providing elongated ridges having said swellable properties according to the invention allows to provide that have a higher adhesion between the two structured substrates than what can be achieved by devices of prior art that only propose arrays of protrusions that have very tiny dimensions. Furthermore, the device of the invention having elongated ridges with the described swelling properties allow to achieve reduced time to achieve a good adhesion, an easier assembly procedure and a less demanding alignment. This makes the device and its method of use much more practical in surgical practices compared to methods and devices proposed in the prior art. Preferably, the device of the invention relies on the use of bio-absorbable and/or degradable materials. More preferably, the bioresorbable material is a composite bioresorbable material.

In an embodiment said plurality of ridges are linear-shaped ridges arranged according to concentric squares or rectangles. Adapting the shape of the arrangement of ridges allows to adapt the form in function of the apertures that have to be closed and sealed.

In an embodiment said plurality of ridges are curved-shaped ridges. Curved shaped ridges can be more tolerant to alignment issues as well as deformation and folding of the substrates during surgery procedure. Curved shapes having continuous curvature and no ridges can also provide a better hermeticity in these cases.

In an embodiment said curved-shaped ridges are arranged according to concentric circles or ellipses. Arranging ridges in concentric circles or ellipses can provide an optimum structuration of the portion of the surface interlocking when pressed to each other when closing respectively circular and elliptical apertures.

A concentric arrangement of the interlock elements increases the area of the interlocking element allows to facilitate the manipulation during the operation and ease the alignment and clipping of the two structured layers. In particular, the two substrates can interlock to each other if their centers are facing each other whatever their in-plane orientation is.

In another advantageous embodiment said at least one of said curved-shaped ridges are arranged according to a spiral shape. Using spiral shaped arrangement allows to adapt the attachment and/or peeling force gradually from the center to the border of the arrangement of ridges. It allows to interlock the ridges facing each other by applying a local pressure in a continuous spiral movement, starting from each center and going outwards or the opposite.

In an embodiment said at least two of said plurality of ridges arranged according to different arrangements, preferably a rectangular or square arrangement and a circular or spiral arrangements, or may have a polynomial shape.

In an embodiment said at least a portion of said first plurality of structures and/or at least a portion of said second plurality of structures have different swellable properties. This enables having different adhesion and/or adhesion kinetics in different areas of the devices. This is useful to position the medical device and interlock it partially in a first phase in first areas, to eventually adjust its position before continuing interlocking the two surfaces in other areas. Swelling degree depends nature of the composite material used, and of different composition and/or thickness of the swellable material and the possible superposition of different layers.

In an embodiment said at least a portion of said plurality of ridges have non-uniform swellable properties. Using non-uniform ridges allows to have a higher design flexibility in function of the required attachment and/or peeling forces.

In an embodiment said swellable properties consist in a swelling in the presence of humidity (liquid water or water containing fluids) and wherein said plurality of ridges present a difference in the Young modules of more than 30 MPa, more preferably 300 MPa and even more preferably 3000 MPa.

In an embodiment said plurality of ridges present a difference in change of volume between a dry state and a swelling of less than 20%, preferable less than 10%, even more preferably less than 5%.

In an embodiment the number of said first and/or said second structures is greater than 5, preferably greater than 20, even more preferably greater than 100, possibly greater than 200.

In an embodiment said wherein the maximal width of the ridges, defined relative to said first or said second planes is smaller than 1000 μm, preferably smaller than 500 μm, more preferably smaller than 300 μm, even more preferably smaller than 200 μm.

In embodiments, the cross sections of said ridges, defined orthogonal to said substrates, may have different shapes chosen among:
straight-shaped cross-sections,
curved-shaped cross-sections,
conical cross-sections,
cross sections presenting overhangs that may have any detailed shape of the overhang part.

In an embodiment, the cross-section of said ridges is homogeneous over the length of the ridges, with less than 10% variation in the profile geometry over portions of said ridges.

In an embodiment, the overlapping width, defined in said first and said second plane, of said first structures with said second structures of said second substrates it is facing, when pressed in contact is smaller than 50 μm, or smaller than 30 μm, or smaller than 20 μm, more preferably smaller than 15 μm, or smaller than 10 μm, or more preferably smaller than 5 μm. Said overlapping width is defined prior to the swelling

In an embodiment said the peel off force between interlocked said first and said second substrates is higher than 10N/cm², preferably after a swelling time of less than 2 hours.

In an embodiment said first plurality of structures and/or said second plurality of structures comprises at least two alignment structures for aligning said first and second substrates. At least one of the alignment structures may be an aperture. In an embodiment said first plurality of structures and/or said second plurality of structures comprises at least two conical-shaped apertures for guiding said two alignment protrusions.

The invention is also achieved by a method of fabrication of the medical device.

The method comprises the steps of:
providing an overhang structure mold comprising a plurality of structures, at least a portion of said plurality of structures being a plurality of ridges having each a length that is at least 5 times greater than their width. The width and the length are defined in a plane parallel to said first and/or second surface, said ridges have at least two different cross-sections defined in planes parallel to said plurality of structures;
realizing a composite layer by dispensing a precise amount of a swellable material, made at least partially of PEG-PLA, and preferably comprising a solvent, dispensed on said overhang structure mold;
separating the first substrate from the overhang structure mold, the medical device comprising at least a first plurality of structures. At least a portion of said first plurality of structures is a plurality of ridges having each a length that is at least 5 times greater than their width, the width and the length being defined in a plane parallel to said first and/or second surface, said ridges have at least two different cross-sections defined in planes parallel to said plurality of structures;
providing a medical device comprising two identical first substrates.

In an embodiment, said dispensed swellable material comprises a solvent and wherein said separation is realized after evaporation of the solvent.

In embodiments, the medical device comprises a second substrate, having a different shape than said first substrate, and made with a different overhang structure mold.

The invention is also achieved by a use of the medical device to close an aperture in a thin layer of human connective tissue in a surgical operation wherein suturing is either not possible or cosmetically not appropriate.

### BRIEF DESCRIPTION OF THE DRAWINGS

Further details of the invention will appear more clearly upon reading the following description in reference to the appended figures:

FIG. 1 shows a perspective view of a device of the invention comprising ridges that have a length L at least 5 time greater that their maximal width;

FIG. 2 illustrates a vertical cross section of two pluralities of interlocking ridges before swelling;

FIG. 3 illustrates a vertical cross-section and a top view of two pluralities of interlocking ridges after swelling. The top view illustrates the projected overlapping areas projected in a plane of the device in its interlocked position;

FIG. 4 illustrates a perspective vertical cross section illustration two pluralities of interlocking ridges with a portion of the ridges in the interlocked position;

FIG. 5 illustrates a perspective vertical cross section illustrating two pairs of substrates with pluralities of interlocking ridges. The figures illustrate a situation with a portion of the ridges in the interlocked position and partially connected by a central structural element;

FIG. 6 illustrates a mechanical assembly arranged to peel of an interlocked device from its closed position;

FIG. 7 illustrates a top view on a plurality of ridges of a device of the invention;

FIG. 8 illustrates a top view on complementary plurality of ridges on a second substrate, to be locked to the plurality of ridges of FIG. 6;

FIG. 9 illustrates a gap between two opposing ridges before their swelling;

FIG. 10 illustrates, horizontal cross sections of the complementary ridges of FIG. 6 and FIG. 7 in their locked-in position, i.e. when the device is in its closed position, with in bold the overlap of the complementary ridges;

FIGS. 11 to 20 illustrate embodiments of vertical lateral cross sections of ridges of the device of the invention with varying profiles;

FIGS. 21 and 22 illustrate a realized ridge of a device of the invention, wherein the ridge presents an overhang realized by micro structuring techniques;

FIG. 23 illustrates a picture of a realized part of a device of the invention comprising concentric circular shaped ridges;

FIG. 24 illustrates a spiral shaped ridge;

FIG. 25 illustrates a plurality of ridges arranged according to centered squares;

FIG. 26 illustrates a plurality of ridges arranged according to centered ellipses;

FIG. 27 illustrates a vertical cross-section of the process flow of the manufacturing of one substrate containing a single ridge of the medical device;

FIG. 28 illustrates a device of the invention with a coating applied on the peripheral border to improve hermicity and adhesion to human tissues;

FIG. 29 illustrates an example of fabricated device of the invention with a coating applied on the peripheral border to improve hermicity and adhesion to human tissues;

FIG. 30 illustrates a peel off force measurement of an interlocked device after swelling.

### DETAILED DESCRIPTION AND EMBODIMENTS OF THE INVENTION

The present invention will be described with respect to particular embodiments and with reference to certain drawings, but the invention is not limited thereto. The drawings described are only schematic and are non-limiting. In the drawings, the size of some of the elements may be exaggerated and not drawn on scale for illustrative purposes. The dimensions and the relative dimensions do not correspond to actual reductions to the practice of the invention.

It is to be noticed that the term “comprising” in the description and the claims should not be interpreted as being restricted to the means listed thereafter, i.e. it does not exclude other elements. Also, by “about” or “approximately” in relation to a given numerical value, it is meant to include numerical values within 10% of the specified value. All values given in the present disclosure are to be understood to be complemented by the word “about”, unless it is clear to the contrary from the context.

The indefinite article “a” or “an” does not exclude a plurality, thus should be treated broadly.

Unless defined otherwise, technical and scientific terms used herein have the same meaning as commonly understood by one of ordinary skill in the art to which this invention belongs.

As used herein, the term “compound” referred to substance e.g. biological substance that is composed of one or more materials. In other words, it refers to one or more ingredients which made up a composition. The term “component” as used herein referred to element which is made up of one or several parts e.g. mechanical parts. In other words, the component can be a part that combines with other parts to be functioned or to form something bigger, or to render a specific function when work together with other components.

Reference throughout the specification to “an embodiment” means that a particular feature, structure or characteristic described in relation with the embodiment is included in at least one embodiment of the invention. Thus, appearances of the wording “in an embodiment” or, “in a variant”, in various places throughout the description, are not necessarily all referring to the same embodiment, but several. Furthermore, the particular features, structures or characteristics may be combined in any suitable manner, as would be apparent to a skilled person from this disclosure, in one or more embodiments. Similarly, various features of the invention are sometimes grouped together in a single embodiment, figure or description, for the purpose of making the disclosure easier to read and improving the understanding of one or more of the various inventive aspects. Furthermore, while some embodiments described hereafter include some, but not other features included in other embodiments, combinations of features if different embodiments are meant to be within the scope of the invention, and from different embodiments. For example, any of the claimed embodiments can be used in any combination. It is also understood that the invention may be practiced without some of the numerous specific details set forth. In other instances, not all structures are shown in detail in order not to obscure an understanding of the description and/or the figures.

The invention is mainly, but not exclusively related to chirurgical devices.

As used herein, the term “bioresorbable material” refers to a material that can be bio absorbed by the human body when implanted over an extended period of time. The term “bioresorbable composite material” refers to a material that contains at least 2 different chemical molecules and that can be bio absorbed by the human body when implanted over an extended period of time. Preferably at least one of the bioresorbable material used is swellable when exposed to water or to aqueous solutions.

The term “overhang” is defined broadly as any structure or layer that is wider than its support portion 5, for example a “T-shaped” structure or the like.

The invention consists of new medical devices with textured surface as dural substitutes to get self-sticking property that provides strong adhesion by means of interlocking of two pluralities of structure made of a material with swellable property, so that they stick firmly together when interlocked and following a swelling. used in the inlay overlay technique.

The bioresorbable composite material is tailored during the manufacturing step with interlock geometrical elements, especially tailored by structuring its layer in specific 3-dimensional shapes. The specific overhang shape of the structured swellable material in two pluralities of ridges on two complementary substrates is engineered such as to interlock the two structured layers in dry environment and ensure a hermetic bounding through the water-swollen property of the composite material in wet or under-water environment. Preferably the overhang shape is a “T-shape”.

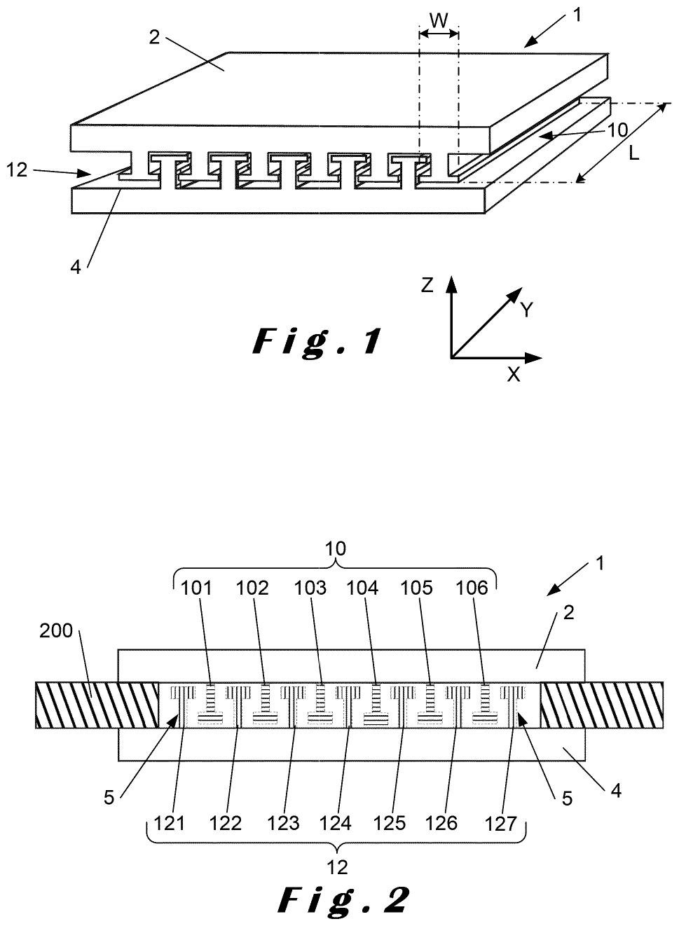

More precisely the invention relates to a medical device 1 for use in surgery comprising at least a first substrate and at least a second substrate, said at least first substrate comprising a first surface, comprising a first plurality of structures, said at least second substrate comprising a second surface, comprising a second plurality of structures, said first and second plurality of structures being arranged to interlock when said first surface is pressed against said second surface and after the swelling of at least one of said first or second plurality of structures, as illustrated in FIGS. 1 and 4.

Swelling is defined by the swelling ration SR: SR=(M_{hydrated}−M_{dehydrated})/M_{dehydrated}.

Herein, M means the mass of the structures that undergo swelling.

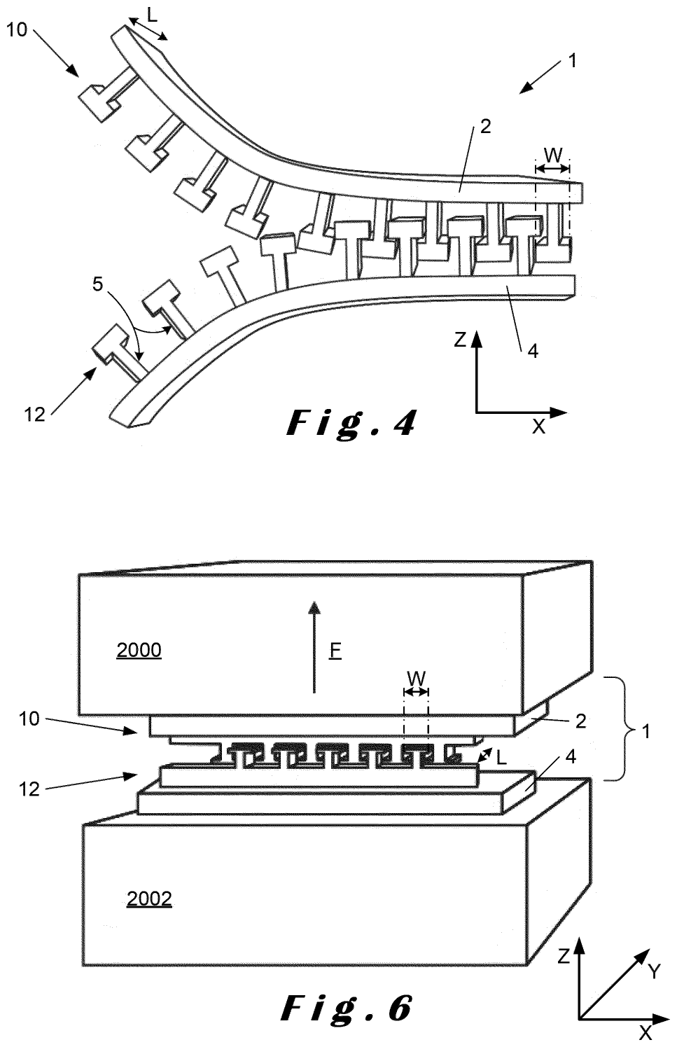

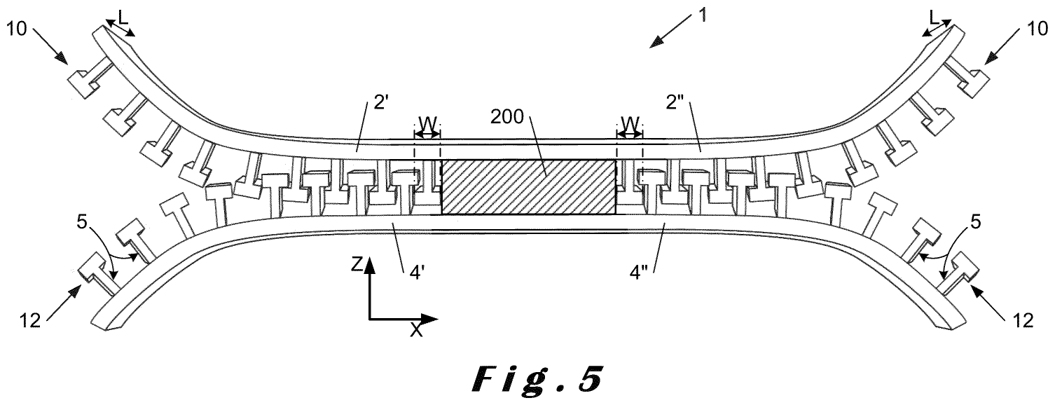

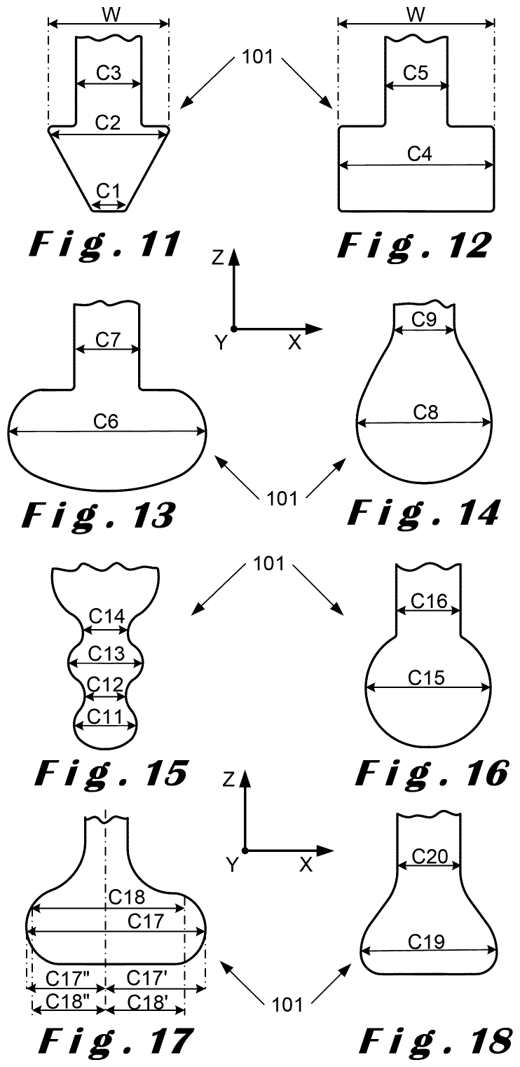

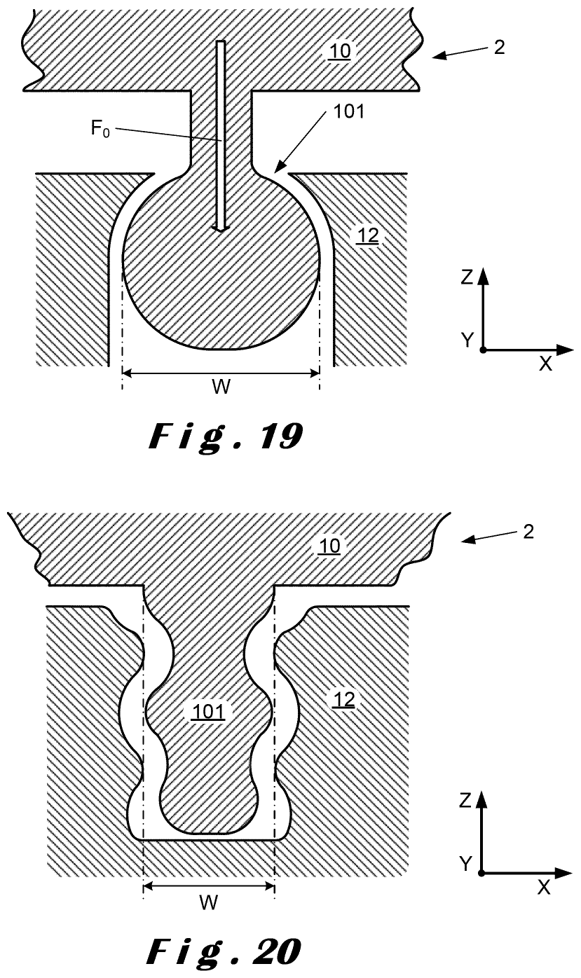

At least a portion of said first plurality of structures 101-106 and/or at least a portion one of said second plurality of ridges 121-127 is a plurality of ridges having each a length L that is at least 5 times greater than their width W, said width W being defined in a plane parallel to said first and/or second surface, as illustrated in FIG. 4-5. It is understood that in some structures, such as the ones illustrated in FIG. 11 or 18 the structures may have different sections having different widths In such cases the width W is referred as the maximal width of the structure, for example the width of section C13 in FIG. 15. Said plurality of structures 101-106 and plurality of ridges 121-127 can have similar or identical geometries, the respective naming plurality of structures and plurality of ridges are used for clarity.

Said first plurality of ridges 101-106 and/or at least one of said second plurality of ridges 121-127 have swellable properties in presence of a liquid and/or vapor. In an embodiment a gas may be introduced in the liquid to modify the swelling kinetics or swelling ratio, or to improve the substrate adhesive properties.

In embodiments the swelling of the ridges 101-106, 121-127 may be realized or accelerated by varying the pH, and/or the temperature. For example, for a pH greater than 10 a swelling of more than 200% in volume may be obtained. It is referred herein to the following publication: https://akinainc.com/polyscitech/products/aquagel/AquaGel-pH.php.

In an embodiment said at least one of said first plurality of structures 101-106 and/or at least one of said second plurality of ridges 121-127 have at least two different cross-sections (C1-C20) defined in at least two virtual surfaces parallel to said first, respectively said second surfaces.

In embodiments, illustrated in the FIGS. 11-20, the cross sections, defined orthogonal to said substrates, may have different shapes chosen among:
straight-shaped cross-section (FIGS. 9, 12),
curved-shaped cross-sections (FIGS. 13, 14, 16-20),
conical cross-section, (FIG. 11),
cross sections presenting overhangs that may have an overhang part comprising different sags (FIGS. 15, 20),
cross section may be asymmetrical comprising different lateral radii (C17′, C17″, C18′, C18″) as illustrated in FIG. 17.

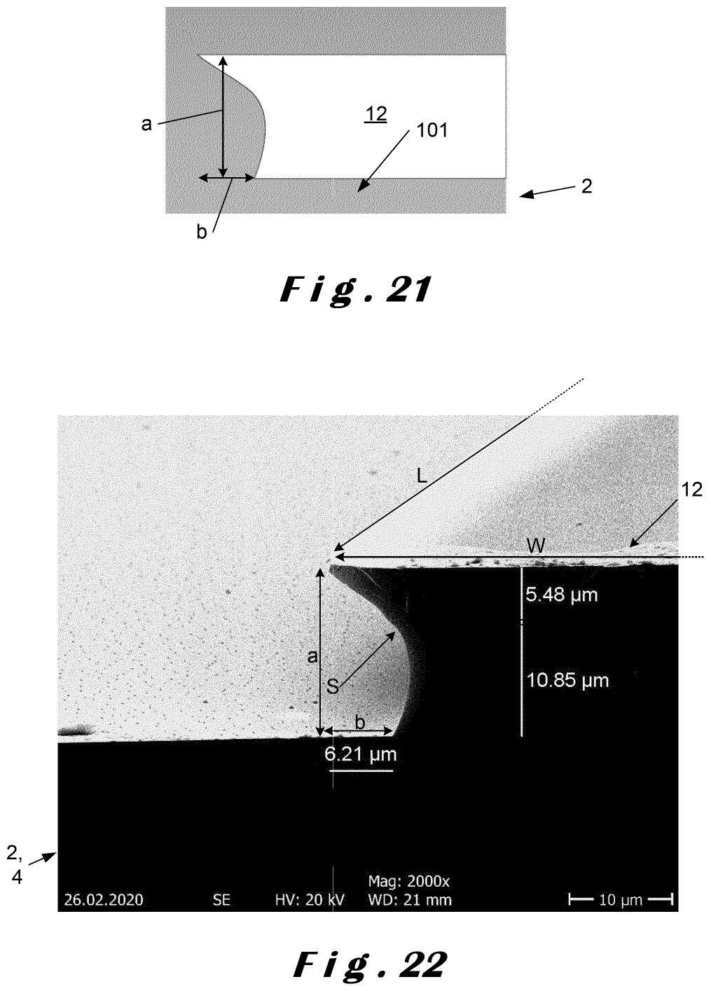

FIG. 21 and FIG. 22 shows a typical lateral cross section of a ridge 101 having a height a and an overhang b. FIG. 22 shows a realized ridge wherein the structure has a first curved section having a height of 10.85 μm and a second curved section having a height of 5.48 μm, the overhang width b being about 6.21 μm. The details shape S of the cross section of an overhang structure, as illustrated in FIGS. 21 and 22 may be defined by a polynomial. In an embodiment, said plurality of ridges are linear-shaped ridges arranged according to concentric squares or rectangles.

In an embodiment said plurality of ridges are curved-shaped ridges 101-106, 121-127.

In an embodiment said curved-shaped ridges 101-106, 121-127 are arranged according to concentric circles or ellipses.

Preferably, at least one of said first and second substrates 2, 4 are essentially made of bioresorbable material, preferably thin, with a thickness below 1 mm, and are preferably at least partially stretchable. As examples of substrate materials, organic materials such as polymer containing biocompatible materials are preferred. As examples, graft materials made of bovine or equine collagen sources, or synthetic material such as Polyethylene terephthalate (PET), Polyurethane (PU), and more preferably synthetic and resorbable such as Polylactic acid (PLA), Polyglycolide (PGA), poly-caprolactone (PLC) can be used. Preferably both said first and second substrates are essentially made of bioresorbable materials. Essentially here refers to material composition being bioresorbable at least 90% by weight, preferably at least 95% by weight, more preferably at least 99% by weight bioresorbable. Indeed, impurities in the material cannot be avoided fully, either due to their manufacturing processes leaving some residues or due to the presence of additives in low amount in the bioresorbable materials, additives being not fully bioresorbable but being difficult to replace by bioresorbable alternatives.

In an embodiment, the first substrate 2, respectively the second substrate 4 and the first plurality of structures 101-106 and/or respectively said second plurality of structures 121-127 are made of the same bioresorbable material or material composite. In this case, said substrate and plurality of structures are said to have a homogeneous material composition. Alternatively, different bioresorbable materials or material composites can be used for the substrates and its respective plurality of structures/ridges.

In embodiments, the medical device 1 comprises more than one interlockable substrates as illustrated in FIG. 5. FIG. 5 illustrates a variant in which two pairs of opposite substrates 2′,4′, 2″, 4″ are connected by a central structural element 200. Such structural element 200 is preferable a flexible element that may be configured as a pivoting axis such as a flexible layer extending over at least a portion of a length of the substrates 2′,4′, 2″, 4″. In variants the structural element 200 may be connected to all the substrates 2′,4′, 2″, 4″, or to two of them. For example, the structural element 200 may be a flexible mechanical connection between two first substrates 2, 2′ or between two second substrates 4′, 4″ or between a first substrate 2′, 2″ and an opposite second substrate 4, 4′.

In an embodiment (not illustrated) the medical device may be realized by a single mold and from a single first substrate 2 that comprises two portions that are separated by a joining section. Upon unmolding such a first substrate may be folded by about 180° according to a virtual folding defined in said joining section. The folding operation may lead that the two portions are separated. In order to facilitate that separation operation, the folding area may comprise a series of apertures that may be through-apertures allowing for an operator to separate, if needed, the two portions of the first substrate and provide the two elements of the medical device.

In variants, the area of the folding axis is a thinner area than the rest of the first substrate so that the substrate becomes separated in two separate portions by the disruption of the mechanical weak folding area, by the simple folding force or by a slight lateral pulling force.

In variants, not illustrated, a plurality of first and second substrates may be assembled and interconnected by pivoting axes or flexible elements or bridges so that the medical device can be applied onto a round surface to be covered and healed.

It is understood that first and second substrates may have any shape, such as a circular, elliptical or rectangular shape.

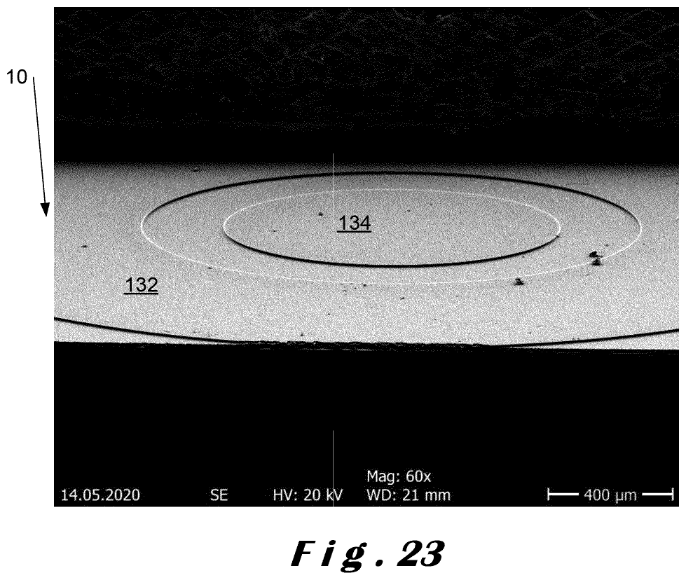

To increase the area of bonding, the interlock element of the invention may be engineered and organized in concentric arrangement with a simple axis of rotation to facilitate the manipulation during the operation and ease the alignment and clipping of the two layers. This is illustrated in FIGS. 23, 26.

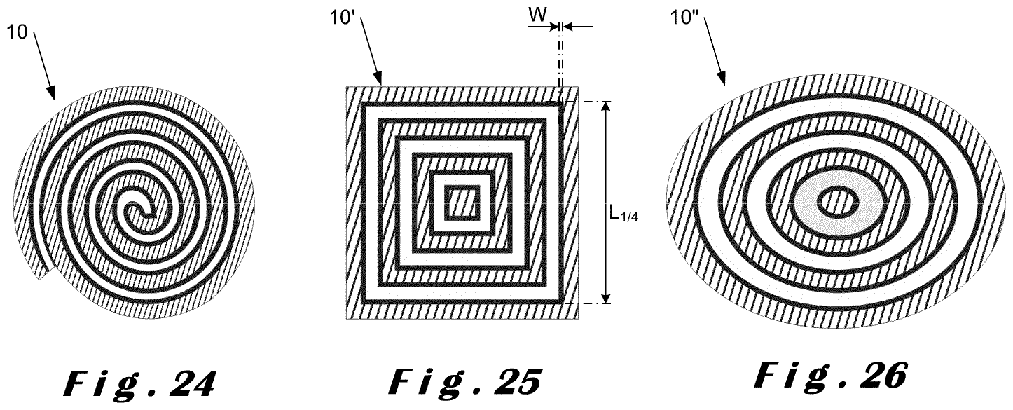

In another advantageous embodiment said at least one of said curved-shaped ridges are arranged according to a spiral shape (FIG. 24).

In an embodiment said at least two of said plurality of ridges arranged according to different arrangements, preferably a rectangular or square arrangement and a circular or spiral arrangement.

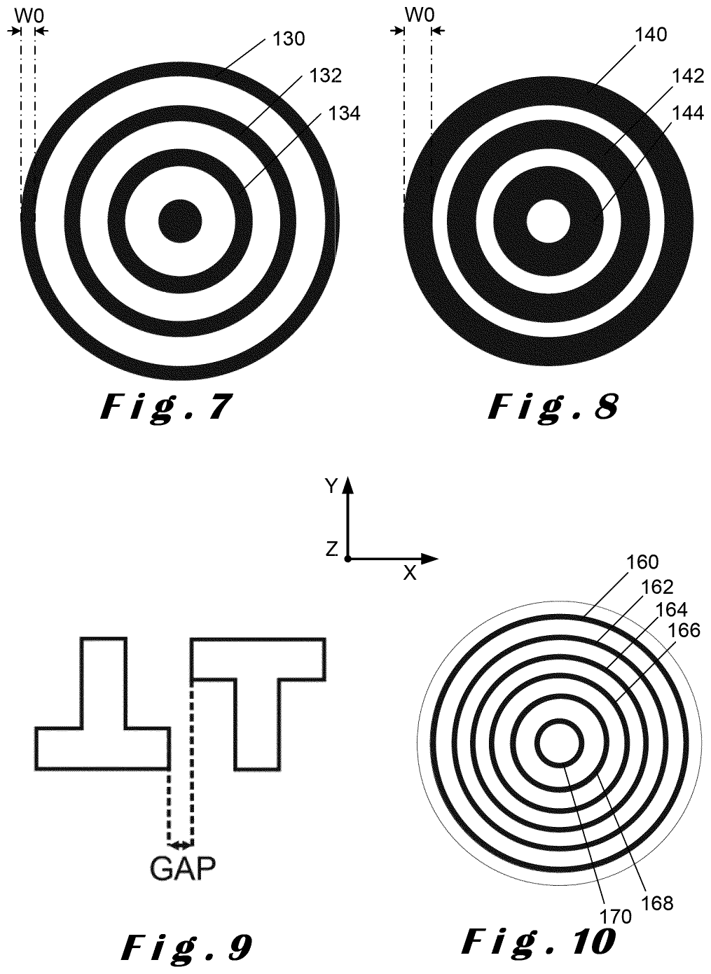

FIG. 7 illustrates a top view on a first concentric arrangement of circular-shaped ridges 130, 132, 134 of a device 1 of the invention.

FIG. 8 illustrates a top view on a second concentric arrangement of circular-shaped ridges 140, 142, 144 on a second substrate, to be locked to the plurality of ridges 130, 132, 134 of FIG. 7.

In exemplary executions the concentric ridges of the embodiment of FIG. 7 and FIG. 8 may have the following (table 1) combination of typical dimensions of widths of the ridges and their projected gaps. The gaps are defined as the projected aperture between opposing structures before their assembly and swelling so that the stick together after swelling.

TABLE 1
Typical widths of a first 101-106 and second 121-127 array of concentric
ring ridges and the typical gap between them after assembly.

| First concentric | Second ridge | Gap between first and of |
|---|---|---|
| ridge width | width W | second concentric ridges |
| W[microns] | [microns] | [microns] |
| 150 | 250 | 0 |
| 150 | 250 | ±5 |
| 150 | 250 | ±10 |
| 550 | 1050 | 0 |
| 550 | 1050 | ±5 |
| 550 | 1050 | ±10 |

The ridges comprise preferably a base 5, illustrated in FIGS. 2, 5 that may have a height, defined orthogonal to its local support area, that is greater or smaller or equal than said with W. In an embodiment said at least a portion of said first plurality of ridges 101-106 and/or at least a portion of said second plurality of ridges 121-127 have different swellable properties.

In an embodiment said at least a portion of said plurality of ridges 101-106, 121-127 have non-uniform swellable properties.

In an embodiment said swellable properties consist in a swelling in the presence of humidity and wherein said plurality of ridges 101-106, 121-127, present a difference in the Young modules of more than 5%, preferably more than 10%, even more preferably more than 20%.

In an embodiment said plurality of ridges 101-106, 121-127 present a difference in elongation between a dry state and a swollen state of more than 5%, preferably more than 10%, even more preferably more than 20%.

In an embodiment said the number of said first and/or said second structures is greater than 5, preferably greater than 20, even more preferably greater than 100 or even greater than 200.

In an embodiment said wherein the maximal width of said plurality of ridges 101-106, 121-127, defined relative to said first or said second planes is smaller than 1000 μm, preferably smaller than 500 μm, more preferably smaller than 300 μm.

In an embodiment said the overlapping width W0, defined in said first and said second plane, of said first structures and/or said second structures when pressed in contact is smaller than 50 μm, preferably smaller than 30 μm, more preferably smaller than 15 μm, even more preferably smaller than 10 μm, even more preferably smaller than 5 μm.

In an embodiment the peel off force between interlocked said first and said second substrates is higher than 10N/cm².

In embodiments the adhesion between the structures may be improved by a coating chosen among collagen, fibrin, gelatin or a combination of such coatings.

In an embodiment said first plurality of ridges 101-106 and/or said second plurality of ridges, 121-127 comprises at least two alignment protrusions for aligning said first and second substrates.

In an embodiment said first plurality of ridges 101-106 and/or said second plurality of ridges 121-127 comprises at least two conical-shaped apertures for guiding said two alignment protrusions.

In a second aspect the invention relates to a method of fabrication of the medical device 1.

To define the optimized shape of the medical device 1, the selection is based by design iterations essentially considering manufacturing limitation aspects such as the aspect ratio (width versus height of the structure) undercut size to ensure good molding without structure breaks during the demolding phases. An optimal bounding area and the mechanical and swelling behavior of the composite material are taken in consideration.

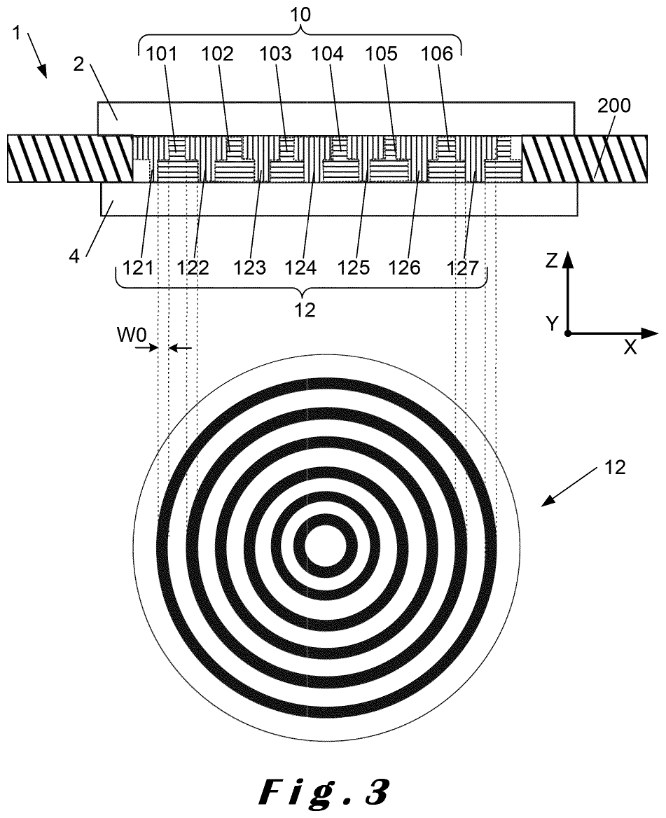

For example, based on iterative manufacturing trials the shape of an advantageous geometry of the medical device 1 is fixed as illustrated in the FIG. 3:
the cross-section of the interlocking element 101-106, 121-127 has a height of 15 microns,
the cross-section of the interlocking element 101-106, 121-127 has an overhanging width of 5-7 microns.

Simulation of the performance of the interlocking features 101-106, 121-127 have been performed by finite element analysis with the software Comsol Multiphysics.

For the theoretical model a mesh with node elements was designed. The geometry was constructed based on the manufactured sample architecture. The material used for the study is a co-polymer material containing polyethylene glycol (PEG) and polylactide (PLA). Table 2 show the material properties (it is referred here to the material data sheet) used as input parameters.

TABLE 2
Young's modulus, tensile strength and swelling
in water of a bioresorbable material, in this example
a composite co-polymer material containing polyethylene
glycol (PEG) and polylactide (PLA).

| Range | Low | Typical | High |
|---|---|---|---|
| Young's modulus | 2500 | 3500 | 4500 |
| (MPa) | | | |
| Tensile strength | 50 | 70 | 100 |
| Swelling (%) | 1 | 3 | 8 |
| Young's modulus | 2500 | 3500 | 4500 |
| (MPa) | | | |
| Tensile strength | 50 | 70 | 100 |
| Swelling (%) | 1 | 3 | 8 |

The invention is also achieved by a method of fabrication of the medical device 1 as described hereafter.

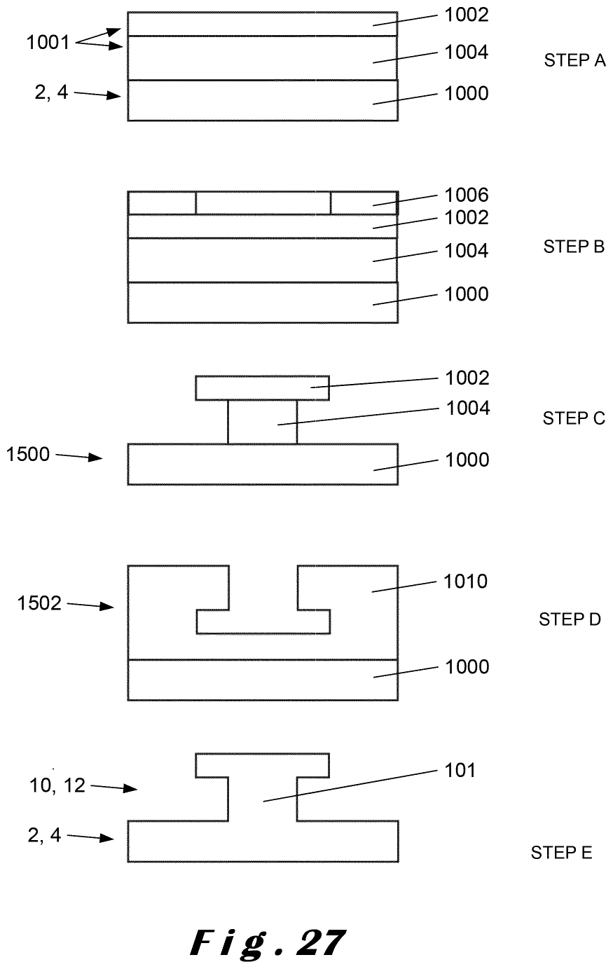

The required interlocking geometrical elements of the device 1 is preferably manufactured by realizing a template matrix by photolithography. The process steps are illustrated in FIG. 27.

The main process steps are the following:

Step A: coating of layers 1004 and 1002:
spin-coating of a bottom layer 1004 of lift-off resist being a polymeric based on polymethylglutarimide (PMGI) having a high developability in an aqueous alkaline solution;
baking of the PMGI layer 1004 alone is used to dry and remove the solvent contained in the PMGI layer using a hot plate and by heating slowly at 220° C. in 30-40 minutes;
depositing on top of said bottom layer 1004 a top layer 1002 consisting of polymeric negative tone photoresist sensitive in the range of the i-line (360 nm);
baking of the bottom and the top layers 1004, 1002 using a hot plate and preferably by heating 5 minutes at 100° C.;

STEP B: patterning the layer stack 1001 comprising the bottom and top layers 1004, 1002 by contact photolithography to expose the pattern on the top layer 1002;

STEP C developing in an aqueous alkaline solution and removing selectively the top layer 1002 and the bottom layer 1004 to form the template matrix 1500 comprising “T” shaped structures on said glass substrate 1000;

STEP D realizing an overhang structure mold 1502, defined also as mold or overhang mold, consisting in:
pouring a precise amount of a visquous liquid on said first template matrix to obtain layer 1010 with a predetermined thickness;
curing of said viscous liquid by heating 2 hours at 80° C., or by applying an electromagnetic irradiation such as UV light, so as to obtain the overhang structure mold 1502 having a complementary structure of the structures of said template matrix 1500. After curing, the viscous liquid becomes an elastomeric layer, preferably made of polydimethylsiloxane (PDMS);
separation of the overhang structure mold 1502 from said first template matrix 1500;

STEP E realizing the ridges 101-106, 121-127 of the medical device 1 by:
realizing a composite layer 101 by dispensing a precise amount of a swellable material, preferably made, at least partially of PEG-PLA, and preferably comprising a solvent, directly dispensed on said overhang mold 1502 to obtain the desired thickness of the two components 2, 4 of the medical device 1;
removing, the medical device 1 from the overhang mold 1502.

In an embodiment, the removal step is realized after evaporation of a solvent, if a solvent is present. In the embodiment in which a swellable bioresorbable material is used as a major component of the medical patch 1, such as a PEG-PLA composite, it is preferred to use a solvent to dissolve the material and shape it before evaporation. Using a solvent is preferred in the removal step of the fabrication, because thermal shaping methods such as thermoforming, injection molding and hot-embossing are not possible for some materials that will deteriorate at an elevated temperature.

In an embodiment, the composite layer 101, a PEG-PLA composite material is dissolved in Acetone (10% solution).

In an embodiment, the method steps are repeated by providing a second different overhang structured mold having structures that are complementary to a first overhang structured mold 1502.

Alternatively, one can provide both the first and second substrates after cutting a larger foil in which layout are incorporated in parallel both first and second substrate layouts. Adaptation of the described process steps for other materials or material composites being bioresorbable can be implemented by the man skilled in the art.

In another embodiment, that can be combined with various other embodiments as described herein the device of the invention may include a sealing coating or sealing layer applied on the peripheral border of said first and/or second substrates. Typically, the sealing coating or sealing layer is made of a bioabsorbable surgical sealants which is FDA approved. The aim a sealing coating or layer is to enhance the fixing and adhesion of the patch to the human tissues in contact with it, to increase the strength of the seal, improve the water-tight sealing and hermeticity, and promote the tissue growth.

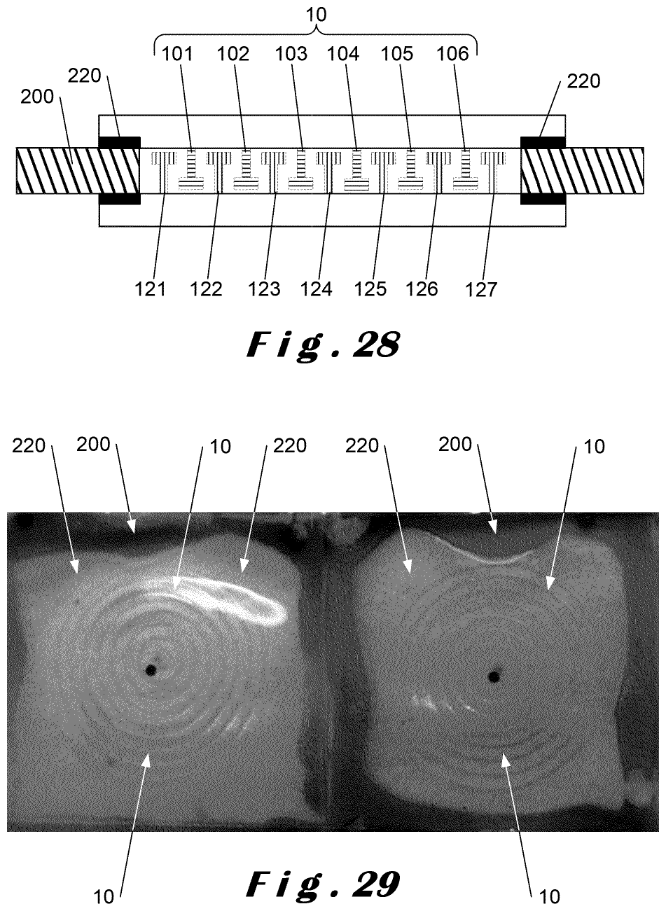

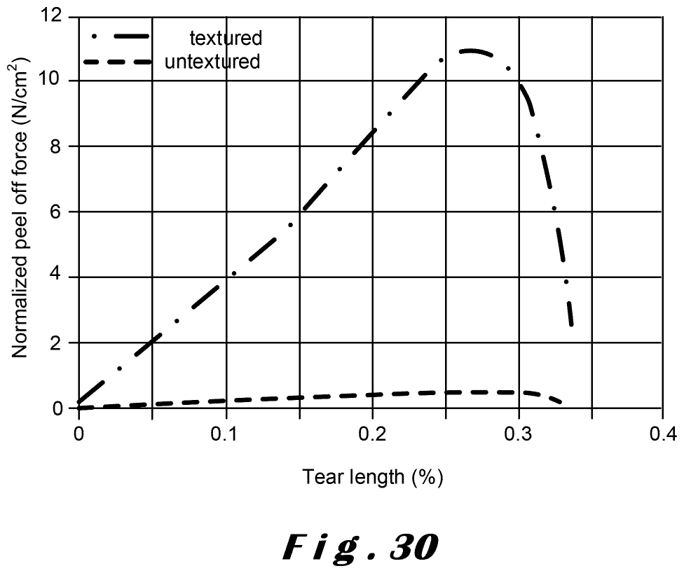

When applied, such sealing coating has experimentally proven to increase the hermeticity of the patches of a factor 10. As an example, blue PEG-NH2 hydrogel can be applied to the edges of the PEG-PLA composite patch, with various coating techniques, either during the patch manufacturing of just prior to its use. Different types of bioabsorbable surgical sealants are available on the market, made of synthetic material such as PEG-NH2-NHS hydrogel or of fibrin and albumin prepared from human source, or could be a combination of both types of sealants. FIG. 28 illustrates such a device of the invention with a coating 220 applied on the peripheral border to improve hermicity and adhesion to human tissues. The coating 220 may be applied to both sides of the structural element 200 or to a single side.

FIG. 29 shows a portion of a realized batch of PEG-PLA patches 1 produced with a coating made of PEG-NH2 hydrogel applied on the edges manually with a syringe.

### Alignment Features

The interlock element 1 is preferably organized in concentric arrangement. Each array of concentric circles has an interlock array structure having the opposite configuration. For the experimental study circles with different widths W and gap tolerance were evaluated and are summarized in the Table 2 (further paragraph experimental results).

Additional alignment helping features can be present on the medical devices 1. For example, printed moiré or Vernier pattern on said first and said second substrate can provide a visual help to perform the alignment of both substrates. For example, bio-absorbable blue ink concentric complementary circle on both substrates can provide a visual guidance of the alignment process.

Additionally, other printed patterns can interfere and provide a distinctive pattern, for example a moiré pattern, when in close contact. This can provide a visual indication of having the first and second substrates in contact and there at least partially locked.

Other visual non printed features can be implemented on the two substrates 2, 4 to provide indications of alignments and proximity of the two substrates 2,4. As example, micro or nanostructure can provide variable optical effect related to the alignment and proximity of structures located on both substrates. Other features can be implemented to help alignment with imaging tools in the visible, infrared, terahertz spectra or with ultrasonic imaging. Parts of the two substrates 2, 4 can be arranged to have a high transparency to help alignment processes.

### Experimental Results:

Devices with different concentric arrangements were realized by using the method described above with PEG-PLA structured material used as such a place of the dural substitute. FIG. 23 shows a top view of 2 ridges 132, 134 of a realized substrate comprising a plurality 10 of ridges.

Values of realized samples are summarized in Table 3. Table 3 shows the values that have been obtained by experimental optimization that consisted in varying the ring dimensions such as their position and ridge widths and dimension and shape of the ridge edge as illustrated on FIGS. 21 and 22.

TABLE 3
this table summarizes geometries that have provided the best results.
They are based on a concentric arrangement of the ridges that have
a thickness of 150 pm for the structures on a first substrate and
450pm for the rings of the structures on the second substrate 4.

| | | Option A | Option B | |
|---|---|---|---|---|
| | | Ridge's edge | Ridge's edge | Option C |
| | Common for | from center | from center | Ridge's edge from |
| | all options | [microns] | [microns] | center [microns] |
| | Ridge width | no gap (in case of | 5 microns gap | 10 microns gap |
| Ridge | (W) | perfect | between two | between two |
| number | [microns] | alignment) | adjacent ridges | adjacent ridges |
| 0 | — | 500 | 495 | 480 |
| 1 | 150 | 650 | 645 | 630 |
| 2 | 400 | 1050 | 1045 | 1030 |
| 3 | 150 | 1200 | 1195 | 1180 |
| 4 | 400 | 1600 | 1595 | 1580 |
| 5 | 150 | 1750 | 1745 | 1730 |
| 6 | 400 | 2150 | 2145 | 2130 |
| 7 | 150 | 2300 | 2295 | 2280 |
| 8 | 400 | 2700 | 2695 | 2680 |
| 9 | 150 | 2850 | 2845 | 2830 |
| 10 | 400 | 3250 | 3245 | 3230 |
| 11 | 150 | 3400 | 3395 | 3380 |
| 12 | 400 | 3800 | 3795 | 3780 |
| 13 | 150 | 3950 | 3945 | 3930 |
| 14 | 400 | 4350 | 4345 | 4330 |
| 15 | 150 | 4500 | 4495 | 4480 |
| 16 | 400 | 4900 | 4895 | 4880 |
| 17 | 150 | 5050 | 5045 | 5030 |
| 18 | 400 | 5450 | 5445 | 5430 |
| 19 | 150 | 5600 | 5595 | 5580 |
| 20 | 400 | 6000 | 5995 | 5980 |

To examine the interlocking behavior and the performance of the devices, the interlocking shear and normal strength were measured with a tensile tester for pull-off and peel tests in wet condition.

For the experiment, two devices with complementary configuration were put into contact with each and expose to water for one hour before the test.

FIG. 6 illustrates a mechanical assembly comprising two supports 2000 arranged to measure the peel-off force of an interlocked device 1 from its closed position. FIG. 28 illustrates the response of the assembly with the PEG-PLA surfaces texturized compared to a reference without surface texturization.

The texturization has demonstrated, the ability to improve by a factor 10 the cohesion of the assembly compared to an untextured one with more than 10N/cm² peel off strength achieved within a short immersion in water (one hour).

### Implementation Examples

The combination of interlocking geometry combined with the intrinsic material property will enhance the performance of dural substitute existing products and thus it is intended to significantly decrease cerebrospinal fluid leakage and post-operative complications and avoid revision surgeries.

The medical device of the invention is preferably sterilized at the end of its manufacturing and/or before its intended use. Different sterilization methods can be applied, such as radiation-based sterilization with gamma ray or E-beam radiation, or chemical-based sterilization. Radiation-based sterilization creates a risk of modifying the polymer chain length and structures, or to further crosslinked polymer, resulting in modified mechanical properties. For these reasons, chemical sterilization is preferred with for examples exposure to ethylene oxide (EO) or preferably to vaporized hydrogen peroxide (VHP).

Chemical sterilization using EO and VHP may be suitable for polymers if they can withstand a short exposure to slightly elevated temperatures under moist conditions. However, EO exposure can also have a dramatic effect on the morphology of the polymeric implants and alter the swelling properly of the polymer used in the fabrication of the patch. These detrimental effects can potentially be circumvented with VHP, which was not found to alter the morphology of porous polymer scaffolds. Other sterilization methods known to the man skilled in the art may be used to obtain a sterilized medical device (1) for use in surgery.

The concept of modifying surface properties of implantable materials may also serve as a platform technology in other surgical disciplines.

Peritoneal repair surgery (such as hernia repair or any other situation where closure of the abdominal wall is needed), especially during minimally invasive surgery with laparoscopy where surgical suturing is extremely difficult to handle and very time consuming. As the use of minimally invasive surgery is increasing, the need for self-closing devices are gaining a lot of importance.

Pleura repair surgery. Similar to peritoneal repair, minimally invasive surgical techniques such as thoracoscopy are on the rise. After each surgery the pleura needs to be closed in a water- and air-tight manner.

Surgery of the oral or nasal cavity where suturing is impossible, e.g. repair of mucous membranes, repair of the eardrum, reconstruction of the palate.

Vascular surgery: any situation where larger blood vessels need to be closed such as in bypass surgery or vascular reconstruction surgery. Additionally, vascular substitute materials (which are usually made of Gore-Tex) could be structured to enhance tightness of closure.

Every situation where an anatomical cavity consisting of a thin wall of connective tissue needs to be closed tightly. Such situations are found in closure of dura mater, peritoneum or pleura.

Every surgical situation where a thin layer of connective tissue needs to be closed and suturing is not possible or cosmetically not appropriate, e.g. pediatric surgery, plastic surgery.

1. A medical device for use in surgery comprising at least a first substrate and at least a second substrate, said at least first substrate comprising a first surface comprising a first plurality of structures, said at least second substrate comprising a second surface comprising a second plurality of structures, said first and second plurality of structures being arranged to interlock when said first surface is pressed, by applying a force Fo, against said second surface,
wherein
at least a portion of said first plurality of structures and/or at least a portion of said second plurality of structures is a plurality of ridges, each of the ridges having a length that is at least 5 times greater than the ridge's width, the width and the length being defined in a plane parallel to said first and/or second surface,
said ridges have at least two different cross-sections defined in planes parallel to said first, respectively said second surface,
said ridges have swellable properties in presence of a liquid and/or vapor.

2. The medical device according to claim 1, wherein the first substrate and second substrate are made, at least partially, of a bioresorbable material having swellable properties and high stiffness.

3. The medical device according to claim 2, wherein said bioresorbable material is a composite bioresorbable material.

4. The medical device according to claim 1, wherein said plurality of ridges are linear-shaped ridges arranged according to concentric squares or rectangles.

5. The medical device according to claim 1, wherein said plurality of ridges are curved-shaped ridges.

6. The medical device according to claim 5, wherein said curved-shaped ridges are arranged according to concentric circles or ellipses.

7. The medical device according to claim 5, wherein at least one of said curved-shaped ridges is arranged according to a spiral shape.

8. The medical device according to claim 1, wherein at least two of said plurality of ridges are arranged according to different arrangements chosen among rectangular, square, circular, spiral, polynomial shapes.

9. A medical device according to claim 1, wherein at least two portions of said ridges have different swellable properties.

10. A medical device according to claim 1, wherein at least a portion of said plurality of ridges has non-uniform swellable properties.

11. A medical device according to claim 1, wherein said swellable properties consist in a swelling in the presence of humidity and wherein said plurality of ridges present a Young's modulus of more than 30 MPa.

12. A medical device according to claim 1, wherein said plurality of ridges present a swelling, in presence of a liquid and/or vapor, of less than 20.

13. A medical device according to claim 1, wherein the number of said ridges is greater than 5.

14. A medical device according to claim 1, wherein the maximal width of said plurality of ridges is smaller than 1000 μm, said widths being defined relative to said first or said second planes.

15. A medical device according to claim 1, wherein the overlapping width, defined in said first and said second plane, of said ridges, when pressed in contact, is smaller than 50 μm.

16. A medical device according to claim 1, wherein the peel-off force F between interlocked first and said second substrates is higher than 10N/cm².

17. A medical device according to claim 1, wherein said first plurality of structures and/or said second plurality of structures comprises at least two alignment protrusions for aligning said first and second substrates.

18. A medical device according to claim 17, wherein said first plurality of structures and/or said second plurality of structures comprises at least two conical-shaped apertures for guiding said two alignment protrusions.

19. A medical device according to claim 1, wherein said at least first substrate and said at least second substrate are bonded in at least one bond area for aligning said first and second substrates.

20. A method to fabricate the medical device according to claim 1, comprising the steps of:
providing an overhang structure mold comprising a plurality of structures of which at least a portion consists in a plurality of ridges, each of the ridges having a length that is at least 5 times greater than the ridge's width, the width and the length being defined in a plane parallel to said first and/or second surface, said ridges having at least two different cross-sections defined in planes parallel to said plurality of structures,
realizing a composite layer by dispensing, on said overhang structure mold, a precise amount of a swellable material made at least partially of PEG-PLA, and providing on said overhang structure mold a first substrate comprising a first surface comprising a first plurality of structures,
separating the first substrate from the overhang structure mold to provide a first substrate comprising at least a first plurality of structures at least a portion of said first plurality of structures being a plurality of ridges, each of the ridges having a length that is at least 5 times greater than the ridge's width, the width and the length being defined in a plane parallel to said first and/or second surface, said ridges having at least two different cross-sections defined in planes parallel to said plurality of structures,
providing a medical device comprising at least one first substrate.

21. The method according to claim 20, wherein said medical device comprises two of said first substrates.

22. The method according to claim 21, wherein a first substrate comprises two portions that are linked by a joining portion and wherein the two substrates of the medical device are provided by the folding and/or cutting of said two portions according to a folding and/or cutting axis defined in said joining portion.

23. The method according to claim 20, wherein said medical device comprises a second substrate, made with a different overhang structure mold, and having a different shape than said first substrate.

24. The method according to claim 20, wherein said dispensed swellable material comprises a solvent and wherein said separation is realized after evaporation of the solvent.

25. A method for performing a surgical operation, the method comprising providing the medical device according to claim 1, and utilizing the medical device to close an aperture in a thin layer of human connective tissue, wherein either suturing is not possible or cosmetically not appropriate.
